(19) United States
(12) Patent Application Publication
(10) Pub. No.: US 2025/0262445 A1
(43) Pub. Date: Aug. 21, 2025

(54) WEARABLE MEDICAL DEVICE IMPLEMENTING LOW POWER COMMUNICATION

(71) Applicant: West Affum Holdings DAC, Dublin (IE)

(72) Inventor: Mark Moore, Kirkland, WA (US)

(73) Assignee: West Affum Holdings DAC, Dublin (IE)

(21) Appl. No.: 19/031,937

(22) Filed: Jan. 18, 2025

Related U.S. Application Data
(60) Provisional application No. 63/622,690, filed on Jan. 19, 2024.

(51) Int. Cl. A61N 1/39; H04W 4/80

(52) U.S. Cl. CPC A61N 1/3904; A61N 1/3925; H04W 4/80

(57) ABSTRACT

The disclosure teaches systems and techniques to improve power management in a Wearable Medical Device. Various embodiments are disclosed that implement low-power wide area network solutions that reduce power consumption while also maintaining communication between the Wearable Medical Device and outside entities, such as a remote patient data platform. Additional embodiments are disclosed that accomplish communication between a Wearable Medical Device and external entities by implementing a read/write memory within a removable component of the Wearable Medical Device.

### CROSS-REFERENCE(S) TO RELATED APPLICATION(S)

This application claims the benefit of U.S. Provisional Application No. 63/622,690, filed Jan. 19, 2024, the disclosure of which is incorporated herein by reference in its entirety.

### BACKGROUND

Often, a patient may need nearly continuous assistance of a medical device, such as in cases where the patient may have a serious cardiac condition. However, such patients are also often ambulatory, thus requiring that the medical device be mobile while in operation so that the patient can walk or move around. A wearable medical device (“WMD”) is an advanced form of such a medical system. A WMD typically includes one or more wearable components that a patient can wear or carry, and possibly other components that can be portable, or stationary such as base station and/or an electric charger. The WMD may also include one or more associated software packages, such as software applications (“apps”), which can be hosted by the wearable component, and/or by a mobile device, and/or by a remote computer system that is accessible via a communications network such as the internet, and so on.

A WMD typically includes a sensor that can sense when a parameter of the patient is problematic, and cause the WMD to initiate an appropriate action. The appropriate action could be for the WMD to communicate with the patient or even with a bystander, to transmit an alert to a remotely located clinician, and to even administer treatment or therapy to the patient by itself. A WMD may actually include more than one sensor, which may sense more than one parameter of the patient. The multiple parameters may be used for determining whether or not to administer the treatment or therapy, or be suitable for detecting different problems and/or for administering respectively different treatments or therapies to the patient.

A WMD may also include the appropriate components for implementing a wearable cardioverter defibrillator (“WCD”), a pacer, and so on. Such a WMD can be for patients who have an increased risk of sudden cardiac arrest (“SCA”). In particular, when people suffer from some types of heart arrhythmias, the result may be that blood flow to various parts of the body is reduced. Some arrhythmias may result in SCA, which can lead to death very quickly, unless treated within a short time, such as 10 minutes. Some observers may have thought that SCA is the same as a heart attack, but it is not. For such patients, an external cardiac defibrillator can deliver a shock through the heart, and restore its normal rhythm. The problem is that it is hard for an external cardiac defibrillator to be brought to the patient within that short time. One solution, therefore, is for such patients to be given a WMD that implements a WCD. This solution is at least temporary and, after a while such as two months, the patient may instead receive a surgically implantable cardioverter defibrillator (“ICD”), which would then become a permanent solution.

A WMD that implements a WCD typically includes a harness, vest, belt, or other garment that the patient is to wear. The WMD system further includes additional components that are coupled to the harness, vest, or other garment. Alternately, these additional components may be adhered to the patient's skin by adhesive. These additional components include a unit that has a defibrillator, and sensing and therapy electrodes. When the patient wears this WMD, the sensing electrodes may make good electrical contact with the patient's skin and therefore can help sense the patient's Electrocardiogram (“ECG”). If the unit detects a shockable heart arrhythmia from the ECG, then the unit delivers an appropriate electric shock to the patient's body through the therapy electrodes. The shock can pass through the patient's heart and may restore its normal rhythm, thus saving the patient's life.

The subject matter discussed in this Background should not be taken as an acknowledgement or suggestion that such subject matter is prior art or exists as part of the common general knowledge. Rather, the discussion of any subject matter in this section is indicative of the determinations made by the inventors which led to the disclosures presented below. These determinations alone may be inventive.

### SUMMARY OF THE DISCLOSURE

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The disclosure teaches systems and techniques to improve power management in a Wearable Medical Device. Various embodiments are disclosed that implement low-power wide area network solutions that reduce power consumption while also maintaining communication between the Wearable Medical Device and outside entities, such as a remote patient data platform. Additional embodiments are disclosed that accomplish communication between a Wearable Medical Device and external entities by implementing a read/write memory within a removable component of the Wearable Medical Device.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a conceptual diagram of a patient wearing an exemplary WMD system, made according to embodiments;

FIG. 2 is a diagram showing sample components of an external defibrillator, made according to embodiments;

FIG. 3 is a diagram of sample embodiments of components of a WMD system made in accordance with this disclosure;

FIG. 4 is a conceptual block diagram of a Wearable Medical Device environment in which embodiments are implemented;

FIG. 5 is a functional flow diagram illustrating a process for a WMD system to report its location using LPWAN; and

FIG. 6 a functional flow diagram illustrating a process for a WMD system to transmit patient and/or WMD information to the RPDP using LPWAN.

### DETAILED DESCRIPTION

Illustrative embodiments are illustrated in the attached figures and will be described below. It should be understood that changes can be made to the illustrative embodiments without departing from the spirit and scope of the claimed subject matter. Alternative embodiments of the disclosure may be implemented without some or all of the specific details described below. In some instances, well-known circuits, structures, and techniques have not been shown to avoid obscuring the understanding of this description.

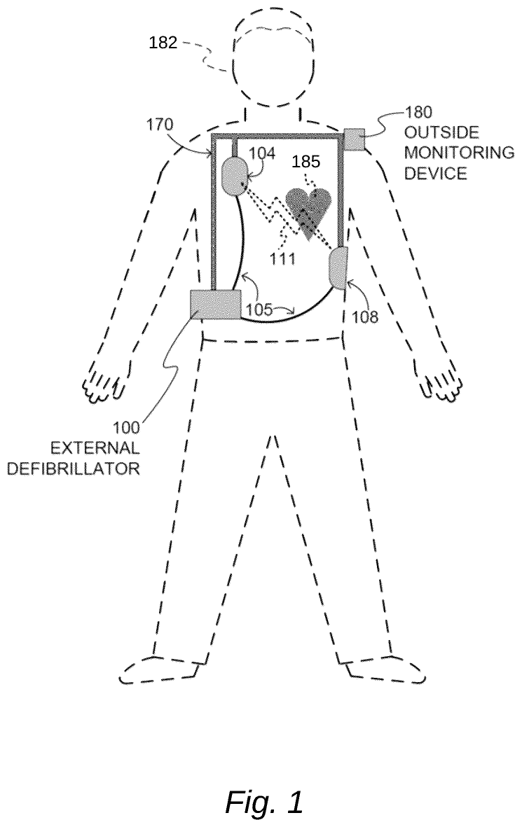

FIG. 1 depicts an exemplary WMD system being worn by a patient 182, according to embodiments of the present disclosure. In this particular example, the WMD system includes a Wearable Cardioverter Defibrillator (WCD), although in other embodiments the WMD system could incorporate any other type of wearable medical device, such as a wearable ECG monitor, a medical alert system, an advanced fitness tracker, or the like. Patient 182 may also be referred to as a person and/or wearer since the patient is wearing components of the WMD system. While patient 182 may be a “user” of the WMD system, this is not a requirement. For instance, a user of the WMD system may also be a clinician such as a doctor, nurse, emergency medical technician (EMT) or other similarly tasked individual or group of individuals. In some cases, a user may even be a bystander. The particular context of these and other related terms within this description should be interpreted accordingly.

A WMD system according to embodiments can be configured to defibrillate the patient who is wearing the designated WMD system. Defibrillating can be accomplished by the WMD system delivering an electrical charge to the patient's body in the form of an electric shock. The electric shock can be delivered in one or more pulses.

FIG. 1 also depicts components of a WMD system made according to embodiments. One such component is a support structure 170 that is wearable by patient 182. Accordingly, support structure 170 is configured to be worn by patient 182 for at least several hours per day, and for at least several days, even a few months. It will be understood that support structure 170 is shown only generically in FIG. 1, and in fact partly conceptually. FIG. 1 is provided merely to illustrate concepts about support structure 170 and is not to be construed as limiting how support structure 170 is implemented, or how it is worn.

Support structure 170 can be implemented in many different ways. For example, it can be implemented in a single component or a combination of multiple components. In embodiments, support structure 170 could include a vest, a half-vest, a garment, etc. In such embodiments such items can be worn similarly to analogous articles of clothing. In embodiments, support structure 170 could include a harness, one or more belts or straps, etc. In such embodiments, such items can be worn by the patient around the torso, hips, over the shoulder, etc. In embodiments, support structure 170 can include a container or housing, which can even be waterproof. In such embodiments, the support structure can be worn by being attached to the patient's body by adhesive material, for example as shown and described in U.S. Pat. No. 8,024,037. Support structure 170 can even be implemented as described for the support structure of US Pat. App. Publ′n No. US2017/0056682, which is incorporated herein by reference in its entirety for all purposes. Of course, in such embodiments, the person skilled in the art will recognize that additional components of the WMD system can be in the housing of a support structure instead of being attached externally to the support structure, for example as described in the US2017/0056682 document. There can be other examples.

The WMD system illustrated in FIG. 1 includes an external defibrillator 100. As described in more detail later in this document, some aspects of external defibrillator 100 include a housing and an energy storage module within the housing. As such, in the context of a WMD system, defibrillator 100 is sometimes called a main electronics module. The energy storage module can be configured to store an electrical charge. Other components can cause at least some of the stored electrical charge to be discharged via electrodes through the patient to deliver one or more defibrillation shocks through the patient.

FIG. 1 also illustrates sample defibrillation electrodes 104, 108, which are coupled to external defibrillator 100 via electrode leads 105. Defibrillation electrodes 104, 108 can be configured to be worn by patient 182 in a number of ways. For instance, defibrillator 100 and defibrillation electrodes 104, 108 can be coupled to support structure 170, directly or indirectly. In other words, support structure 170 can be configured to be worn by patient 182 to maintain at least one of electrodes 104, 108 on the body of patient 182, while patient 182 is moving around, etc. The electrode can be thus maintained on the body by being attached to the skin of patient 182, simply pressed against the skin directly or through garments, etc. In some embodiments the electrode is not necessarily pressed against the skin but becomes biased that way upon sensing a condition that could merit intervention by the WMD system. In addition, many of the components of defibrillator 100 can be considered coupled to support structure 170 directly, or indirectly via at least one of defibrillation electrodes 104, 108.

When defibrillation electrodes 104, 108 make good electrical contact with the body of patient 182, defibrillator 100 can administer, via electrodes 104, 108, a brief, strong electric pulse 111 through the body. Pulse 111 is also known as shock, defibrillation shock, therapy, electrotherapy, therapy shock, etc. Pulse 111 is intended to go through and restart heart 185, in an effort to save the life of patient 182. Pulse 111 can further include one or more pacing pulses of lesser magnitude to simply pace heart 185 if needed, and so on.

A defibrillator typically decides whether to defibrillate or not based on an ECG signal of the patient. However, external defibrillator 100 may initiate defibrillation, or hold-off defibrillation, based on a variety of inputs, with the ECG signal merely being one of these inputs.

A WMD system according to embodiments can obtain data from patient 182. For collecting such data, the WMD system may optionally include at least an outside monitoring device 180. Device 180 is called an “outside” device because it could be provided as a standalone device, for example not within the housing of defibrillator 100. Device 180 can be configured to sense or monitor at least one local parameter. A local parameter can be a parameter of patient 182, or a parameter of the WMD system, or a parameter of the environment, as will be described later in this document.

For some of these parameters, device 180 may include one or more sensors or transducers. Each of such sensors can be configured to sense a parameter of patient 182, and to render an input responsive to the sensed parameter. In some embodiments the input is quantitative, such as values of a sensed parameter; in other embodiments the input is qualitative, such as informing whether a threshold is crossed, and so on. Sometimes these inputs about patient 182 are also referred to herein as physiological inputs and patient inputs. In embodiments, a sensor can be construed more broadly, as encompassing many individual sensors.

Optionally, device 180 is physically coupled to support structure 170. In addition, device 180 may be in operative communication with other components that are coupled to support structure 170. Such communication can be implemented by a communication module, as will be deemed applicable by a person skilled in the art in view of this description.

In embodiments, one or more of the components of the shown WMD system may be customized for patient 182. This customization may include several aspects. For instance, support structure 170 can be fitted to the body of patient 182. For another instance, baseline physiological parameters of patient 182 can be measured, such as the heart rate of patient 182 while resting, while walking, motion detector outputs while walking, etc. The measured values of such baseline physiological parameters can be used to customize the WMD system, to make its diagnoses more accurate, since patients' bodies differ from one another. Of course, such parameter values can be stored in a memory of the WMD system, and so on. Moreover, a programming interface can be made according to embodiments, which receives such measured values of baseline physiological parameters. Such a programming interface may input automatically in the WMD system these, along with other data.

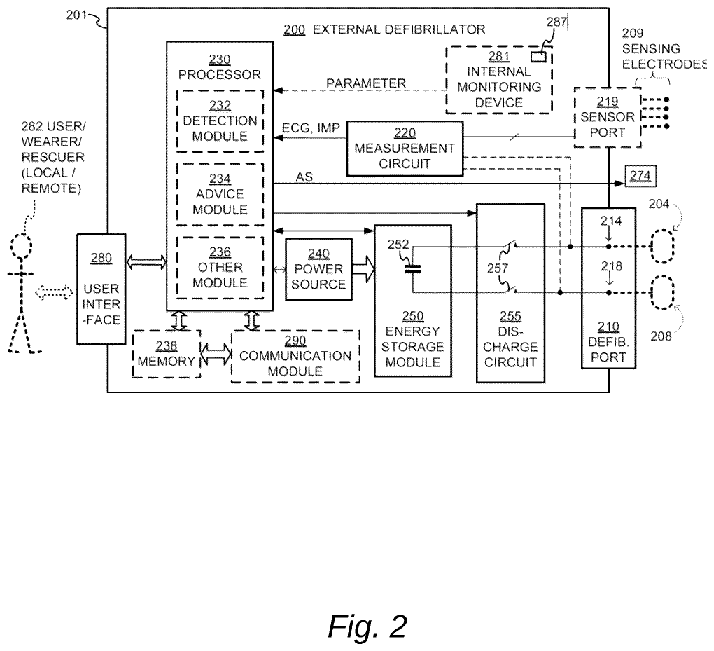

FIG. 2 is a diagram showing components of an external defibrillator 200, made according to embodiments, for use in some WMD systems where a patient has a serious cardiac condition. These components can be, for example, included in external defibrillator 100 of FIG. 1. The components shown in FIG. 2 can be provided in a housing 201, which may also be referred to as casing 201.

External defibrillator 200 is intended for a patient who would be wearing it, such as patient 182 of FIG. 1. Defibrillator 200 may further include a user interface 280 for a user 282. User 282 can be patient 182, also known as wearer 182. Or user 282 can be a local rescuer at the scene, such as a bystander who might assist, or a trained person. Or user 282 might be a remotely located trained caregiver in communication with the WMD system.

User interface 280 can be made in several ways. User interface 280 may include output devices, which can be visual, audible, or tactile, for communicating to a user by outputting images, sounds or vibrations. Images, sounds, vibrations, and anything that can be perceived by user 282 can also be called human-perceptible indications (HPIs). There are many examples of output devices. For example, an output device can be a light, or a screen to display what is sensed, detected and/or measured, and provide visual feedback to rescuer 282 for their resuscitation attempts, and so on. Another output device can be a speaker, which can be configured to issue voice prompts, beeps, loud alarm sounds and/or words to warn bystanders, etc.

User interface 280 may further include input devices for receiving inputs from users. Such input devices may include various controls, such as push buttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock.

Defibrillator 200 may include an internal monitoring device 281. Device 281 is called an “internal” device because it is incorporated within housing 201. Monitoring device 281 can sense or monitor patient parameters such as patient physiological parameters, system parameters and/or environmental parameters, all of which can be called patient data. In other words, internal monitoring device 281 can be complementary or an alternative to outside monitoring device 180 shown in FIG. 1. Allocating which of the parameters are to be monitored by which of monitoring devices 180, 281 can be done according to design considerations. Device 281 may include one or more sensors, as also described elsewhere in this document.

Patient parameters may include patient physiological parameters. Patient physiological parameters may include, for example and without limitation, those physiological parameters that can be of any help in detecting whether the patient is in need of a shock or other intervention or assistance. Patient physiological parameters may also optionally include the patient's medical history, event history and so on. Examples of such parameters include the patient's ECG, blood oxygen level, blood flow, blood pressure, blood perfusion, pulsatile change in light transmission or reflection properties of perfused tissue, heart sounds, heart wall motion, breathing sounds and pulse. Accordingly, monitoring devices 180, 281 may include one or more sensors configured to acquire patient physiological signals. Examples of such sensors or transducers include one or more electrodes to detect ECG data, a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of this disclosure, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, a local parameter is a trend that can be detected in a monitored physiological parameter of patient 282. A trend can be detected by comparing values of parameters at different times over short and long terms. Parameters whose detected trends can particularly help a cardiac rehabilitation program include: (a) cardiac function (e.g. ejection fraction, stroke volume, cardiac output, etc.); (b) heart rate variability at rest or during exercise; (c) heart rate profile during exercise and measurement of activity vigor, such as from the profile of an accelerometer signal and informed from adaptive rate pacemaker technology; (d) heart rate trending; (e) perfusion, such as from SpO2, CO2, or other parameters such as those mentioned above, (f) respiratory function, respiratory rate, etc.; (g) motion, level of activity; and so on. Once a trend is detected, it can be stored and/or reported via a communication link, along perhaps with a warning if warranted. From the report, a physician monitoring the progress of patient 282 will know about a condition that is either not improving or deteriorating.

Patient state parameters may include recorded characteristics of patient 282, such as motion, posture, whether the patient has spoken recently and may be also what the patient said, and so on, plus optionally the history of these parameters. Or, one of these monitoring devices could include a location sensor such as a Global Positioning System (GPS) location sensor. Such a sensor can detect the location, plus a speed can be determined as a rate of change of location over time. Many motion detectors output a motion signal that is indicative of the motion of the detector, and thus of the patient's body. Patient state parameters can be very helpful in narrowing down the determination of whether SCA is indeed taking place.

A WMD system made according to embodiments may thus include a motion detector. In embodiments, a motion detector can be implemented within monitoring device 180 or monitoring device 281. Such a motion detector can be made in many ways as is known in the art, for example by using an accelerometer. In this example, a motion detector 287 is implemented within monitoring device 281. A motion detector of a WMD system according to embodiments can be configured to detect a motion event. A motion event can be defined as is convenient, for example a change in motion from a baseline motion or rest, etc. In such cases, a sensed patient parameter is motion.

System parameters of a WMD system can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if monitoring device 180 or 281 includes a GPS location sensor as per the above, and if it is presumed that the patient is wearing the WMD system.

Defibrillator 200 typically includes a defibrillation port 210, which can be a socket in housing 201. Defibrillation port 210 includes electrical nodes 214, 218. Leads of defibrillation electrodes 204, 208, such as leads 105 of FIG. 1, can be plugged into defibrillation port 210, so as to make electrical contact with nodes 214, 218, respectively. It is also possible that defibrillation electrodes 204, 208 are connected continuously to defibrillation port 210, instead. Either way, defibrillation port 210 can be used for guiding, via electrodes, to the wearer at least some of the electrical charge that has been stored in an energy storage module 250 that is described more fully later in this document. The electric charge will be the shock for defibrillation, pacing, and so on.

Defibrillator 200 may optionally also have a sensor port 219 in housing 201, which is also sometimes known as an ECG port. Sensor port 219 can be adapted for plugging in sensing electrodes 209, which are also known as ECG electrodes and ECG leads. It is also possible that sensing electrodes 209 can be connected continuously to sensor port 219, instead. Sensing electrodes 209 are types of transducers that can help sense an ECG signal, e.g. a 12-lead signal, or a signal from a different number of leads, especially if they make good electrical contact with the body of the patient and in particular with the skin of the patient. As with defibrillation electrodes 204, 208, the support structure can be configured to be worn by patient 282 so as to maintain sensing electrodes 209 on a body of patient 282. For example, sensing electrodes 209 can be attached to the inside of support structure 170 for making good electrical contact with the patient, similarly with defibrillation electrodes 204, 208.

Optionally a WMD system according to embodiments also includes a fluid that it can deploy automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel, so that it does not flow away, after being deployed, from the location it is released near the electrode. The fluid can be used for both defibrillation electrodes 204, 208, and for sensing electrodes 209.

The fluid may be initially stored in a fluid reservoir, not shown in FIG. 2. Such a fluid reservoir can be coupled to the support structure. In addition, a WMD system according to embodiments further includes a fluid deploying mechanism 274. Fluid deploying mechanism 274 can be configured to cause at least some of the fluid to be released from the reservoir and be deployed near one or both of the patient locations to which electrodes 204, 208 are configured to be attached to the patient. In some embodiments, fluid deploying mechanism 274 is activated prior to the electrical discharge responsive to receiving activation signal AS from a processor 230, which is described more fully later in this document.

In some embodiments, defibrillator 200 also includes a measurement circuit 220, as one or more of its working together with its sensors or transducers. Measurement circuit 220 senses one or more electrical physiological signals of the patient from sensor port 219, if provided. Even if defibrillator 200 lacks sensor port 219, measurement circuit 220 may optionally obtain physiological signals through nodes 214, 218 instead, when defibrillation electrodes 204, 208 are attached to the patient. In these cases, the input reflects an ECG measurement. The patient parameter can be an ECG, which can be sensed as a voltage difference between electrodes 204, 208. In addition, the patient parameter can be an impedance, which can be sensed between electrodes 204, 208 and/or between the connections of sensor port 219 considered pairwise. Sensing the impedance can be useful for detecting, among other things, whether these electrodes 204, 208 and/or sensing electrodes 209 are not making good electrical contact with the patient's body. These patient physiological signals may be sensed when available. Measurement circuit 220 can then render or generate information about them as inputs, data, other signals, etc. As such, measurement circuit 220 can be configured to render a patient input responsive to a patient parameter sensed by a sensor. In some embodiments, measurement circuit 220 can be configured to render a patient input, such as values of an ECG signal, responsive to the ECG signal sensed by sensing electrodes 209. More strictly speaking, the information rendered by measurement circuit 220 is output from it, but this information can be called an input because it is received as an input by a subsequent device or functionality.

Defibrillator 200 also includes a processor 230. Processor 230 may be implemented in a number of ways in various embodiments. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 230 may include, or have access to, a non-transitory storage medium, such as memory 238 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

Processor 230 can be considered to have a number of modules. One such module can be a detection module 232. Detection module 232 can include a Ventricular Fibrillation (VF) detector. The patient's sensed ECG from measurement circuit 220, which can be available as inputs, data that reflect values, or values of other signals, may be used by the VF detector to determine whether the patient is experiencing VF. Detecting VF is useful because VF typically results in SCA. Detection module 232 can also include a Ventricular Tachycardia (VT) detector, and so on.

Another such module in processor 230 can be an advice module 234, which generates advice for what to do. The advice can be based on outputs of detection module 232. There can be many types of advice according to embodiments. In some embodiments, the advice is a shock/no shock determination that processor 230 can make, for example via advice module 234. The shock/no shock determination can be made by executing a stored Shock Advisory Algorithm. A Shock Advisory Algorithm can make a shock/no shock determination from one or more ECG signals that are captured according to embodiments and determine whether or not a shock criterion is met. The determination can be made from a rhythm analysis of the captured ECG signal or otherwise.

In some embodiments, when the determination is to shock, an electrical charge is delivered to the patient. Delivering the electrical charge is also known as discharging and shocking the patient. As mentioned above, such can be for defibrillation, pacing, and so on.

In ideal conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in U.S. patent application Ser. No. 16/037,990, filed on Jul. 17, 2018 and since published as US 2019/0030351 A1, and also in U.S. patent application Ser. No. 16/038,007, filed on Jul. 17, 2018 and since published as US 2019/0030352 A1, both by the same applicant and incorporated herein by reference.

Processor 230 can include additional modules, such as other module 236, for other functions. In addition, if internal monitoring device 281 is provided, processor 230 may receive its inputs, etc.

Defibrillator 200 optionally further includes a memory 238, which can work together with processor 230. Memory 238 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. Memory 238 is thus a non-transitory storage medium. Memory 238, if provided, can include programs for processor 230, which processor 230 may be able to read and execute. More particularly, the programs can include sets of instructions in the form of code, which processor 230 may be able to execute upon reading. The programs may also include other information such as configuration data, profiles, scheduling etc. that can be acted on by the instructions. Executing is performed by physical manipulations of physical quantities, and may result in functions, operations, processes, acts, actions and/or methods to be performed, and/or the processor to cause other devices or components or blocks to perform such functions, operations, processes, acts, actions and/or methods. The programs can be operational for the inherent needs of processor 230, and can also include protocols and ways that decisions can be made by advice module 234. In addition, memory 238 can store prompts for user 282 if this user is a local rescuer. Moreover, memory 238 can store data. This data can include patient data, system data and environmental data, for example as learned by internal monitoring device 281 and outside monitoring device 180. The data can be stored in memory 238 before it is transmitted out of defibrillator 200, or be stored there after it is received by defibrillator 200.

Defibrillator 200 can also include a communication module 290, for establishing one or more wired or wireless communication links with other devices of other entities, such as a remote assistance center, Emergency Medical Services (EMS), and so on. The communication links can be used to transfer data and commands. The data may be patient data, event information, therapy attempted, CPR performance, system data, environmental data, and so on. For example, communication module 290 may transmit wirelessly, e.g. on a daily basis, heart rate, respiratory rate, and other vital signs data to a server accessible over the internet, for instance as described in US Pat. Publ′n 20140043149. This data can be analyzed directly by the patient's physician and can also be analyzed automatically by algorithms designed to detect a developing illness and then notify medical personnel via text, email, phone, etc. Module 290 may also include such interconnected sub-components as may be deemed necessary by a person skilled in the art, for example an antenna, portions of a processor, supporting electronics, outlet for a telephone or a network cable, etc.

In specific implementations of embodiments, communication module 290 includes or is a low-power wireless network (LPWAN) transmitter or transceiver. Low-power wireless networks are a key enabler for the Internet of Things (IoT), but familiar options such as Bluetooth, ZigBee, Wi-Fi, or cellular may lack an acceptable combination of extended range and battery life. To address this, sub-GHz LPWAN specifications have been developed, such as LoRaWAN for example. LoRaWAN can achieve up to a 15 km range at power consumption levels low enough to enable 10-year battery life. Further, the availability of development kits let designers quickly deploy a complete LoRaWAN network application with little effort. Accordingly, in one or more embodiments, the WMD may implement an independent LPWAN radio (e.g., LoRaWAN, Helium, etc.) with a CPU running on its own battery Defibrillator 200 may also include a power source 240. To enable portability of defibrillator 200, power source 240 typically includes a battery. Such a battery is typically implemented as a battery pack, which can be rechargeable or not. Sometimes a combination is used of rechargeable and non-rechargeable battery packs. Other embodiments of power source 240 can include an AC power override, for where AC power will be available, an energy-storing capacitor, and so on. Appropriate components may be included to provide for charging or replacing power source 240. In some embodiments, power source 240 is controlled and/or monitored by processor 230.

Defibrillator 200 may additionally include an energy storage module 250. Energy storage module 250 can be coupled to the support structure of the WMD system, for example either directly or via the electrodes and their leads. Module 250 is where some electrical energy can be stored temporarily in the form of an electrical charge, when preparing it for discharge to administer a shock. In embodiments, module 250 can be charged from power source 240 to the desired amount of energy, as controlled by processor 230. In typical implementations, module 250 includes a capacitor 252, which can be a single capacitor or a system of capacitors, and so on. In some embodiments, energy storage module 250 includes a device that exhibits high power density, such as an ultracapacitor. As described above, capacitor 252 can store the energy in the form of an electrical charge, for delivering to the patient.

A decision to shock can be made responsive to the shock criterion being met, as described below. When the decision is to shock, processor 230 can be configured to cause at least some or all of the electrical charge stored in module 250 to be discharged through patient 182 while the support structure is worn by patient 182, so as to deliver a shock 111 to patient 182.

For causing the discharge, defibrillator 200 moreover includes a discharge circuit 255. When the decision is to shock, processor 230 can be configured to control discharge circuit 255 to discharge through the patient at least some of all of the electrical charge stored in energy storage module 250. Discharging can be to nodes 214, 218, and from there to defibrillation electrodes 204, 208, so as to cause a shock to be delivered to the patient. Circuit 255 can include one or more switches 257. Switches 257 can be made in a number of ways, such as by an H-bridge, and so on. Circuit 255 could also be thus controlled via processor 230, and/or user interface 280.

A time waveform of the discharge may be controlled by thus controlling discharge circuit 255. The amount of energy of the discharge can be controlled by how much energy storage module has been charged, and also by how long discharge circuit 255 is controlled to remain open. Defibrillator 200 can optionally include other components.

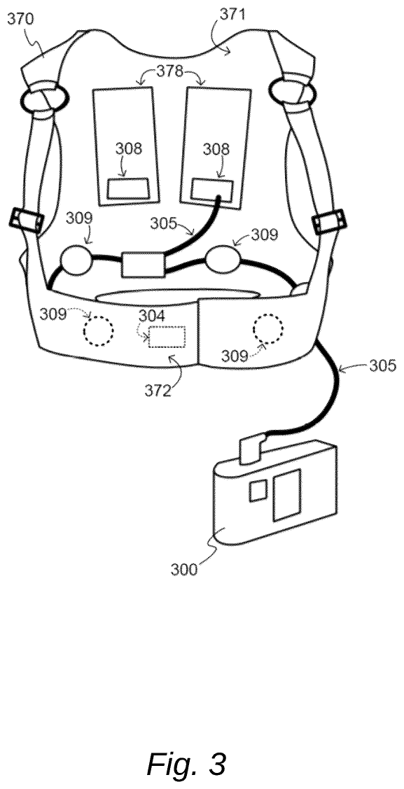

FIG. 3 is a diagram of sample embodiments of components of another exemplary WMD system. In this particular example, a support structure 370 includes a vest-like wearable garment. Support structure 370 has a back side 371 and a front side 372 that closes in front of the chest of the patient.

The WMD system of FIG. 3 may also include an external defibrillator 300 with built-in medical monitoring components. FIG. 3 does not show any support for external defibrillator 300, which may be carried in a purse, on a belt, by a strap over the shoulder, and so on. Wires 305 connect external defibrillator 300 to electrodes 304, 308, 309. Of those, electrodes 304, 308 are defibrillation electrodes, and electrodes 309 are ECG sensing electrodes.

Support structure 370 is configured to be worn by the patient so as to maintain electrodes 304, 308, 309 on a body of the patient. Indeed, back defibrillation electrodes 308 are maintained in pockets 378. Of course, the inside of pockets 378 can be made with loose netting, so that electrodes 308 can contact the back of the patient, especially with the help of the conductive fluid that has been deployed. In addition, sensing electrodes 309 are maintained in positions that surround the patient's torso, for sensing ECG signals and/or the impedance of the patient.

In the nature of a wearable system, ECG signals in a WMD system can sometimes include excessive electrical noise which reduces their usefulness. To ameliorate the problem, multiple ECG sensing electrodes 309 may be provided, for presenting many options to processor 230. These options present additional vectors for sensing the proper ECG signal, thereby reducing the negative impacts of noise.

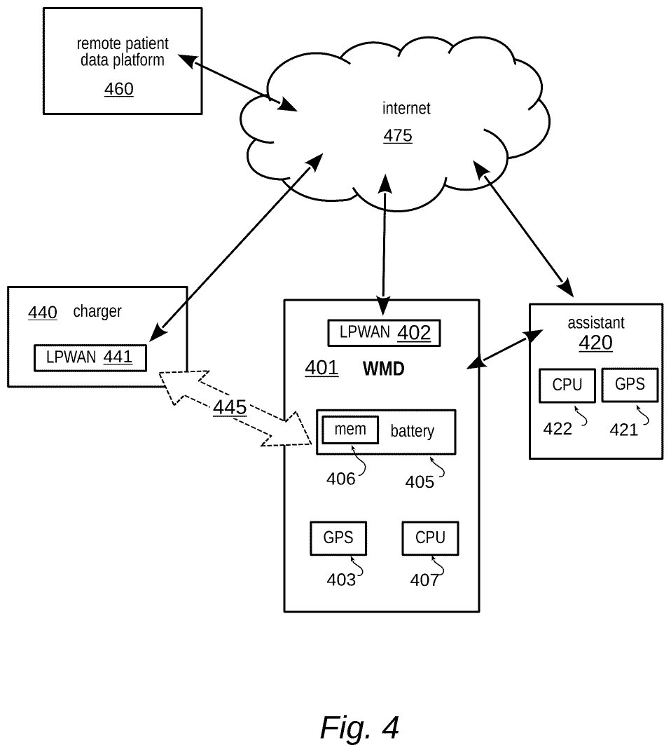

FIG. 4 is a conceptual diagram of yet another WMD system implementing various embodiments of the disclosure. The exemplary WMD system illustrated in FIG. 4 includes a WMD 401, an Assistant 420, a Charger 440, and a Remote Patient Data Platform (RPDP) 460. As shown in FIG. 4, the WMD 401 may be implemented in general accordance with the teachings of the defibrillator 200 illustrated in FIG. 2 and described above. However, the WMD 401 illustrated in FIG. 4 may or may not be a defibrillator and, instead, may be implemented as an alternative wearable medical device monitoring a patient.

In this embodiment, the WMD 401 includes a LPWAN module 402 capable of low-power wireless communication with external devices over a relatively wide area. WMD 401 also includes a GPS sensor 403 capable of determining, using the Global Positioning System, a location of the WMD 401. In addition, the WMD 401 further includes a battery 405 which may further include a memory 406.

The remote patient data platform (RPDP 460) of the exemplary embodiment is a remote patient care platform that accepts patient data from the WMD 401 and presents it to care providers, such as doctors and other medical care specialists. In addition, the RPDP 460 may maintain operational information about WMDs that are subscribed to, or otherwise in operation with, the RPDP 460. That operational information may include location information for each WMD in the system, wear and maintenance information for the WMDs, compliance data for patients in the system, and the like. In one specific implementation, the RPDP 460 may be the CareStation platform owned and operated by Kestra Medical Technologies, Inc.

Various types of WMDs may use what is commonly referred to as an “Assistant,” which is a mobile companion device to provide enhanced functionality to the WMD 401. The exemplary Assistant 420 may be implemented as an app installed on a smartphone or as a special-purpose mobile device. One example of enhanced functionality provided by the Assistant 420 may be to enable data communications between the WMD 401 and the RPDP 460. In these illustrative embodiments, the Assistant 420 may also include its own GPS sensor 421 as described above.

The WMD system may further include a charger 440 for the WMD 401. In these exemplary embodiments, the charger 440 also includes its own LPWAN module 441. In these exemplary embodiments, the charger 440 receives the WMD 401, such as with a docking port, for the purpose of charging the battery 405 of the WMD 401. Such docking port may further include wired communications capabilities 445 while the WMD 401 is docked in the charger 440.

Finally, each of the various components may communicate over a wide area network, such as the Internet 475. Although illustrated as the Internet, it should be appreciated that communications over the wide area network may be encrypted and protected using various known technologies, such as a Virtual Private Network and the like.

The following described embodiments is each implemented using the general WMD system illustrated in FIG. 4 and described above. Some embodiments have minor modifications to that exemplary system. Additional alternative embodiments will also become apparent from the teachings of these exemplary embodiments.

### Integrated Embodiment with Self-Reporting

WMDs are easily lost in the field. Patients wearing them are mobile and may not return the wearable medical devices to the patients' care provider when they are no longer needed. One preferred embodiment of the disclosure incorporates a low power communication system that signals the WMD's location to a central server. In specific implementations, the WMD may signal its location periodically and may do so for extended periods of time.

In this embodiment, the WMD 401 determines its location using location-based technology. Various implementations may use Global Positioning System (GPS) signals, such as through use of an internal GPS receiver 403. In addition, or alternatively, the WMD 401 may triangulate its location through the use of a Wi-Fi receiver (not shown) using the locations and signal strengths of one or more wide area network access points.

In this exemplary embodiment, the WMD 401 implements an independent LPWAN module 402 (e.g., LoRaWAN, Helium, etc.) that includes its own embedded CPU running on its own battery, separate from the CPU 407 and battery 405 of the WMD 401. Such an embodiment allows the location of the WMD 401 to be transmitted to a central server (e.g., RPDP 460) even if the WMD's battery 405 is depleted or not installed. In other implementations, the location of the WMD 401 could be transmitted over a cellular network although at higher cost and power budget.

Additionally, with a communications path between the WMD CPU and the Low power wide area network, that device could be used to send small data sets to the RPDP 460. This embodiment provides some patient data updates for patients who either don't use the Assistant 420 or have a faulty Assistant 420.

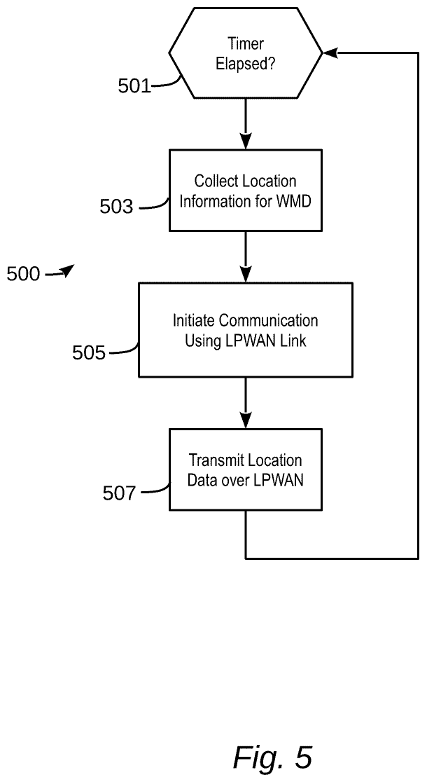

FIG. 5 is a functional flow diagram illustrating a process 500 for a WMD system, such as the WMD system illustrated in FIG. 4 and describe above, to report its location using LPWAN. The process 500 begins at step 501 after a WMD has been prescribed and deployed in the field. At step 501, a timer counts down from a last time when location data for the WMD system was transmitted to the RPDP. Various triggers may cause the timer to be reset, such as transmitting the location of the WMD to the RPDP or for other reasons. For example, a WMD may be docked into a charging station configured to transmit data for the WMD, including the location. However, if the trigger expires, the process 500 proceeds to step 503.

At step 503, the WMD determines its location. In various embodiments, determining the location may be accomplished using a GPS sensor embedded within the WMD. Alternatively, the WMD may triangulate its location using Wi-Fi signals. In yet another alternative, the WMD may estimate its location using, for example, a compass and accelerometer combined with historic location data. Once the WMD location is determined, the process 500 proceeds to step 505.

At step 505, a communication link is established using a LPWAN module between the WMD and a remote location. Advantages of the LPWAN are many, as discussed above, such as enabling a wide area connection using much lower power consumption than Wi-Fi or other wireless technologies. Once the LPWAN link is connected, the process 500 proceeds to step 507.

At step 507, the WMD location is transmitted to a remote location, such as the RPDP, over the LPWAN link. In this way, the location of the WMD may be regularly reported to a monitoring service, such as CareStation, even in the absence of a regular Wi-Fi connection or connected Assistant. The advantages of such a system include the ability to more easily and reliably monitor the locations of each WMD in a WMD system.

### Embodiment with Assistant

In systems where the WMD 401 requires an assistant for remote communication, a working Assistant 420 is required for the WMD 401 to transmit patient data to the RPDP 460. The WMD 401 may also optionally have a Bluetooth radio and supporting software. The WMD 401 may not have direct internet connectivity, so it can use Bluetooth or other short-range wireless communication technology to pass data to the Assistant 420 with internet connectivity. The Assistant 420 then forwards the WMD data to a remote web server, such as the RPDP 460. Without patient data, care givers cannot monitor patients, insurance providers cannot be billed for WMD usage, as well as other complications. However, in WMD systems implementing an Assistant for communications, some patients do not turn on, charge, or carry the Assistant 420.

In this exemplary embodiment, the WMD 401 uses a smart battery 405 with a System on Chip (SOC) that supports I2C, Bluetooth, and internet connectivity (e.g., LPWAN, cellular, etc). The SOC may be powered by the WMD battery 405 or by its own separate battery (not shown). The SOC of this embodiment supports an I2C communications channel at a common address known to the WMD. The WMD 401 sends Bluetooth connectivity data (address, pin, etc.) to the battery 405 over the I2C channel. The SOC may then establish a short-range communication (e.g., BlueTooth) connection to the WMD 401. Once the short-range communication connection is established, the SOC forwards patient data to the RPDP 460. In this exemplary embodiment, the WMD 401 is implemented with LPWAN, and it periodically determines and reports its location using a LPWAN link. In this way, the RPDP 460 may more easily find and track the WMDs, such as at the end of a prescription.

In this exemplary embodiment, the WMD may implement process 500 for determining and reporting the location of the WMD. However, in this exemplary embodiment, the LPWAN module is implemented within the smart batter 405.

### Battery Charger Implemented Embodiment

In WMD systems that include an assistant, not all patients regularly use the Assistant 420. Without the Assistant 420 regularly communicating to the RPDP 460, it may be difficult to determine whether the patient is wearing the WMD 401. Difficulties arise if it cannot be determined that the patient is wearing the WMD. For example, patient compliance cannot be properly monitored, patient data may be incomplete, necessary maintenance items for the WMD may not be provided, agreed billing arrangements may go unfulfilled.

In such an exemplary embodiment, the WMD 401 may not be configured with LPWAN module 402 because WMD 401 relies upon the Assistant 420 for communications. In such an embodiment, the WMD 401 is configured to transmit small amounts of data regularly (e.g., daily) to the RPDP 460 even without use of the Assistant 420.

In one specific implementation, the WMD 401 may be powered by a removable battery 405 with a memory device 406. The WMD is configured to read from and write to the WMD battery 405. The WMD system may also include an external battery charger 440 capable of reading from or writing to the WMD battery's memory device 406.

In such an embodiment, when the battery is in the WMD 401, the WMD 401 records various patient data and/or system data, such as the patient identifier and wear hours for the current wear period along with the dates and times of wear. When the battery 405 is placed in (or connected to) the external charger 440, the external charger 440 reads the recorded patient data and forwards the patient data to the RPDP 460. In an alternative implementation, wireless communication (e.g., Bluetooth or Wi-Fi) between the WMD 401 and the battery charger 440 may be implemented. The charger 440 may then employ LPWAN 441 to wirelessly transmit data to the RPDP 460.

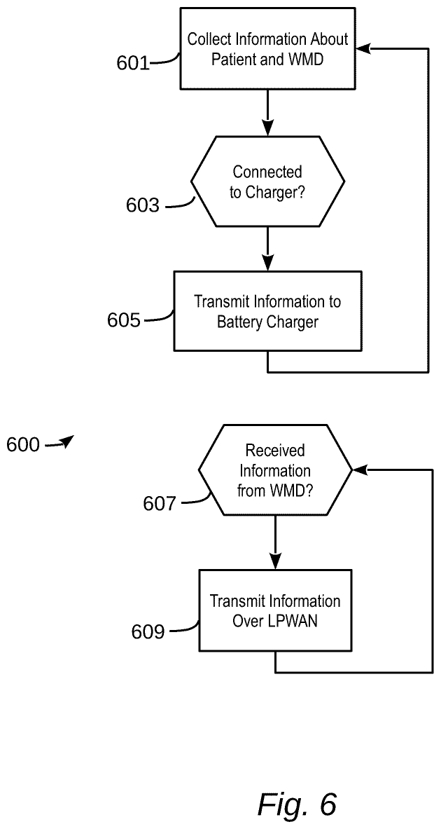

FIG. 6 is a functional flow diagram illustrating a process 600 for a WMD system, such as the WMD system illustrated in FIG. 4 and describe above, to transmit patient and/or WMD information to the RPDP using LPWAN. The process 600 begins at step 601, after a WMD has been prescribed and deployed in the field. At step 601, the WMD captures and stores information about the patient, the WMD, or both. In this exemplary embodiment, the WMD stores the information in a memory within the battery of the WMD.

At step 603, the process 600 monitors for whether the battery has been connected to a charger. In one specific implementation, the battery may be physically removed from WMD and placed into a separate charger. In another specific implementation, the WMD may be connected to the charger such as using a docking port. In another specific implementation, the battery and the charger engage in a short-range wireless connection by virtue of being proximate to each other. Once the process 600 determines that the battery has been connected to the charger, the process 600 continues to step 605.

At step 605, with the battery connected to the charger, the information stored within the memory of the batter is transferred to the charger. The transfer of the information may be through a wired or wireless connection. Once the information has been transferred, the process 600 returns to step 601.

At step 607, the charger determines that it has received the information from the WMD via the battery. As described above, the information about the WMD and/or the patient may have been stored within a memory of the battery and transmitted to the charger upon connection between the battery and the charger.

At step 609, the charger transmits the information about the WMD and/or the patient to the RPDP over a LPWAN link. As described at length, the use of LPWAN provides advantages over existing wireless technologies such as Bluetooth or Wi-Fi. Once the information has been transmitted to the RPDP, the process returns to step 607.

### A Fourth Embodiment

Battery powered medical devices may depend on external communication modules, (e.g. cellular phone, Wi-Fi, Bluetooth, ZigBee, etc.) to send data to a medical reviewing system. Medical device manufacturers typically choose this architecture for two primary reasons. First, it saves battery life for critical medical functions by using low power short range communications (Bluetooth, ZigBee, etc.) rather than Wi-Fi which uses significantly more power. Second, using an external communication module protects the medical device from technological obsolesce. For instance, when cellular network technology changes, the external communication module can be changed/upgraded without having to modify the medical device. Modifying medical devices that require regulatory approval is a non-trivial exercise.

Systems using the foregoing approach rely on the patient or care giver (“user”) to use and maintain the external communications module. If the user does not do so, communications between the medical device and the medical reviewing station can be lost. In such cases, care providers often don't understand how to treat or access the patient. In addition, manufacturers can lose track of where their wearable medical devices are located. These and other problems are prevalent.

In this exemplary embodiment, the user (e.g., patient, care giver, or the like) is prompted to engage with and enable or maintain the external communications module. In scenarios where a WMD has not been in contact with the external communications module for more than a predetermined amount of time, embodiments of the system prompts the user to engage the external communications module. The prompt could be visual or audible (e.g. “Please turn on your device and ensure it is close by”). Other messages could guide the user (e.g., “If your device doesn't turn on, it may need to be charged, see the operating instructions for connecting the charger”).

In various implementations, the frequency of the prompting and even urgency of the prompting could be increased as the time since the last connection increases.

In various implementations, the WMD could also monitor the communication module's battery level and prompt the user to charge the device before the battery is fully drained.

In various implementations, the WMD could prompt to ensure that the communication device/gateway is enabled and/or charged.

In various implementations, the WMD could prompt to ensure that the communication device/gateway is within communication range of the WMD with preemptive prompting to ensure communication device/gateway is charged.

Other embodiments may include combinations and sub-combinations of features described above or shown in the drawings, including for example, embodiments that are equivalent to: providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

plish communication between a Wearable Medical Device and external entities by implementing a read/write memory within a removable component of the Wearable Medical Device

1. A Wearable Medical Device (WMD), comprising:
a support structure configured to be worn by a patient and to support the WMD;
a medical sensor configured to collect patient information while the WMD is being worn by the patient;
a location module configured to determine a location of the WMD; and
a low-power wide-area-network (LPWAN) module configured to enable wireless low-power communications between the WMD and a remote server over a communications link established by the LPWAN module.

2. The WMD recited in claim 1, wherein the WMD comprises a Wearable Cardioverter Defibrillator.

3. The WMD recited in claim 1 further comprising a battery, the WMD being further configured to record data to a memory within the battery, and wherein the battery is configured to communicate with an external battery charger and to transmit the recorded data to the external battery charger.

4. The WMD recited in claim 1, wherein the WMD is further configured to transmit the patient information, the location of the WMD, or both over the communications link.

5. The method for reporting a location of a Wearable Medical Device (WMD), the WMD comprising a support structure and medical monitoring sensors, the WMD being configured to monitor physical parameters of a patient wearing the WMD using the medical monitoring sensors, the method comprising:
determining whether a predetermined time has expired without reporting the location of the WMD;
upon determining that the predetermined time has expired, identifying the location of the WMD by retrieving the location from a location detecting sensor associated with the WMD;
initiating a communication link with a remote server over a low-power wide-area-network module (LPWAN); and
transmitting the location of the WMD to the remote server over the LPWAN communication link.

6. The method recited in claim 1, wherein the location detecting sensor comprises a Global Positioning System component.
